(19) United States
(12) Patent Application Publication
(10) Pub. No.: US 2025/0318933 A1
(43) Pub. Date: Oct. 16, 2025

(54) PATELLAR PROSTHESIS

(71) Applicant: LIMACORPORATE S.P.A., San Daniele Del Friuli (IT)

(72) Inventors: Christoph FIEDLER, Diekhof (DE); Michele PRESSACCO, Martignacco (IT); Massimo CECONI, Travesio (IT)

(21) Appl. No.: 18/866,219

(22) PCT Filed: May 16, 2023
(86) PCT No.: PCT/EP2023/063185

§ 371 Date: Nov. 15, 2024

(30) Foreign Application Priority Data
May 17, 2022 (SM) SM-P-202200207

(51) Int. Cl. A61F 2/38; A61F 2/30

(52) U.S. Cl. CPC A61F 2/3877; A61F 2/30771; A61F 2/3094; A61F 2002/30891; A61F 2002/3092; A61F 2002/30985; A61F 2310/00011

(57) ABSTRACT

A patellar prosthesis includes a support component in metallic material and a joint cap in polymeric material, anchored to an anchoring surface of said support component.

### FIELD OF APPLICATION

The present invention relates to a patellar prosthesis adapted to be fixed to the natural kneecap, or patella, of a patient who must undergo knee arthroplasty.

### BACKGROUND ART

It is known that due to degenerative, congenital or traumatic diseases, or other similar problems, it may be necessary to perform an arthroplasty of the knee joint consisting of the removal of the worn articular surfaces of the femur and tibia and the installation of respective prosthetic femoral and tibial components.

Depending on the degree of knee damage, it is also known to replace the contact surface of the patella facing the artificial joint, installing a patellar prosthesis on the patient's previously shaped natural patella.

Patellar prostheses are known which comprise a monolithic support component, made of metallic material, and a joint cap, made of polymeric material, anchored to the support component and adapted, in use, to be coupled with the intracondylar area of the femoral prosthetic component.

The support component comprises a porous osseointegration layer, a full-body intermediate base, and a further porous layer to which the joint cap is interconnected.

The full-body intermediate base has both the support and separation function between the two porous layers to prevent, in the step of implementation, the injected polymeric material from also filling the pores of the osseointegration layer.

One of the problems of such prostheses is the complexity of making the structures in order to obtain both an effective interconnection of the polymer when being made, and an effective bone regrowth and stability in use.

Currently, the full-body intermediate base is only partially covered by the porous osseointegration layer which is not capable of ensuring a sufficient thickness for bone regrowth. As a result, the prosthesis could fail, i.e., detach from the natural patella.

The porous layer for the polymer interconnection also currently has a problem. In fact, the size of the pores must be specifically envisaged in relation to the density of the polymer which otherwise would not be able to permeate therein. Given that the process conditions, although duly controlled, depend on many environmental factors which cannot be controlled, or on variability in the chemical properties of the polymer and metal materials used to make the prosthesis, it is not uncommon for the interconnection to not occur effectively and therefore be nullified.

Therefore, there is a need to perfect a patellar prosthesis which can overcome at least one of the drawbacks of the prior art.

To do this, it is necessary to solve the technical problem of ensuring the seal between the support component and the joint cap without compromising the characteristic osseointegrative capacity of the reticular structure of the support component.

In particular, an object of the present invention is to make a patellar prosthesis capable of ensuring the maximum possible bone regrowth therein.

Another object of the present invention is to provide a patellar prosthesis in which the joint cap is anchored in a stable, non-removable and simpler manner to the support component.

The Applicant has studied, tested and obtained the present invention to overcome the drawbacks of the prior art, and to obtain the above as well as further aims and benefits.

### DISCLOSURE OF THE INVENTION

The present invention is expressed and characterized in the independent claim. The dependent claims show other features of the present invention or variants of the main solution proposed.

In accordance with the aforesaid objects and to solve the aforesaid technical problem in a new and original manner, also obtaining considerable advantages with respect to the prior art, a patellar prosthesis according to the present invention, comprises a support component in monolithic metallic material and made in a single piece by means of additive manufacturing, provided with an anchoring portion and an interface portion for osseointegration, in direct continuity with said anchoring portion.

A plurality of connection pegs extend from said interface portion.

Said patellar prosthesis further comprises a polymeric joint cap, anchored to said anchoring portion.

Said anchoring portion is fully compact and lacks a porous structure and has an anchoring surface provided with one or more recesses each having anchoring undercuts for said polymeric material.

Furthermore, said interface portion consists of a completely porous structure suitable for osseointegration.

In accordance with another aspect of the present invention, said anchoring surface is curved and has a base area and a raised area between which said recesses extend.

In accordance with another aspect of the present invention, said recesses are delimited by walls defined between said raised area and said base area and which make acute anchoring angles with said base portion.

In accordance with another aspect of the present invention, said recesses comprise a circularly shaped central recess, an annularly shaped peripheral recess which extends around said central recess, and a plurality of radial recesses which extend radially from said peripheral recess.

In accordance with another aspect of the present invention, said radial recesses are angularly spaced according to a constant pitch angle.

In accordance with another aspect of the present invention, said radial recesses communicate on one side with said peripheral recess and on the other side are closed radially.

In accordance with another aspect of the present invention, said radial recesses communicate on one side with said peripheral recess and on the other side are open radially outwards.

In accordance with another aspect of the present invention, said connection pegs have a completely porous structure whereby said connection pegs are completely part of said interface portion.

In accordance with a further embodiment of the present invention, said connection pegs can have an inner reinforcing part, or core, in compact solid material lacking a porous structure which is part of said anchoring portion, and an outer porous part which is part of said interface portion. In such a case, the ratio between a length of said inner reinforcement part and an overall length of said connection peg is between 60% and 100%.

### ILLUSTRATION OF THE DRAWINGS

These and other aspects, features and advantages of the present invention will become clear from the disclosure of some embodiments, given merely by way of non-limiting example, with reference to the attached drawings in which:

FIG. 1 is a schematic view illustrating a patellar prosthesis, according to the present invention, fixed to a patient's natural patella, or kneecap;

FIG. 2 is a side view of the patellar prosthesis of FIG. 1;

FIG. 3 is a sectional view of the prosthesis of FIG. 2;

FIGS. 4-5 are axonometric views of the support component which is part of the patellar prosthesis;

FIGS. 6-7 are views from above and below of FIGS. 4-5;

FIG. 8 is a side view of FIG. 5;

FIG. 9 is a section along the line IX-IX of FIG. 6;

FIGS. 10-11 are embodiment variants of FIGS. 4 and 6.;

FIG. 12 is an axonometric sectional view in which the connection pegs with an only externally porous structure and an internally compact structure are seen.

It should be noted that in the present description the phraseology and terminology used, as well as the figures of the attached drawings also as described, have the sole function of illustrating and better explaining the present invention, having a non-limiting exemplary function of the invention itself, the scope of protection being defined by the claims.

To facilitate understanding, identical reference numbers have been used, where possible, to identify identical common elements in the figures. It should be noted that elements and features of an embodiment can be conveniently combined or incorporated into other embodiments without further clarification.

### DESCRIPTION OF SOME EMBODIMENTS OF THE PRESENT INVENTION

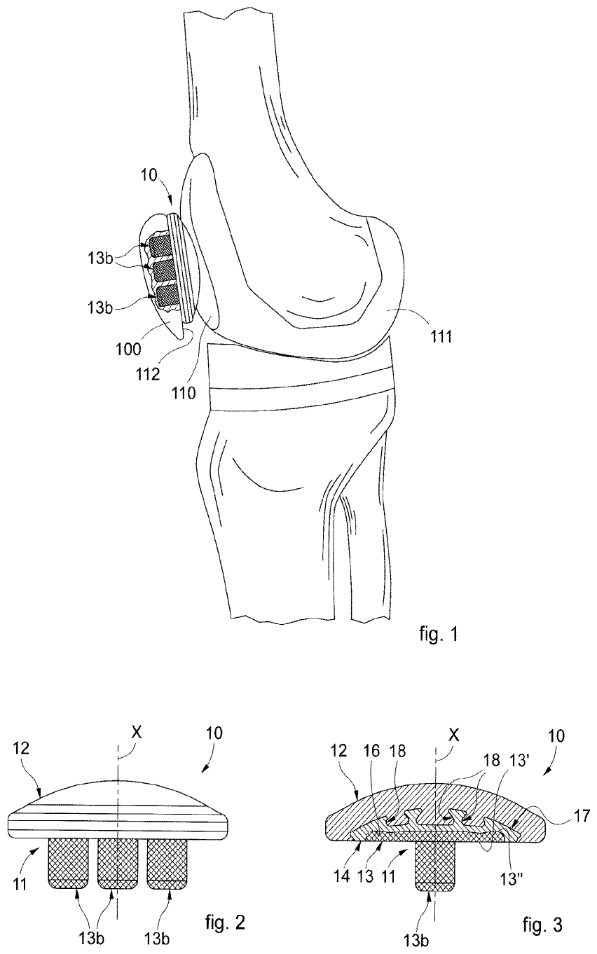

Referring to FIGS. 1-3, a patellar prosthesis 10 according to the present invention can be installed during knee arthroplasty surgery with partial integration of a patient's natural kneecap, or patella 100.

The patellar prosthesis 10 of the present invention comprises a monolithic support component 11, made of metallic material, and a joint cap 12, made of polymeric material, anchored to the support component 11 and adapted, in use, to be coupled with the intracondylar area 110 of a femoral prosthetic component 111, FIGS. 1-3.

The support component 11 comprises an anchoring portion 14 in a compact material, lacking a porous structure and which has an anchoring surface 17 for the polymeric material of the joint cap 12.

Compact material is intended as a structure characterized by the absence of a regular or irregular alternation of solids and voids, as is the case in a reticular structure.

The support component 11 comprises a completely porous interface portion 13 suitable for osseointegration. The interface portion 13 is in direct continuity with the anchoring portion 14.

According to embodiments, the support component 11, which comprises the anchoring portion 14 and the interface portion 13, is made integrally and seamlessly by additive printing technology. Such technology involves scanning successive layers of metallic powder with a high-energy beam in a single continuous process.

A plurality of connection pegs 13b extend from the interface portion 13.

The interface portion 13 has an interface surface 13′ from which said connection pegs 13b extend, which in this case are three.

The interface surface 13′ is substantially flat.

The connection pegs 13b extend along an insertion axis X, which is also an axis of symmetry for the patellar prosthesis 10, on the opposite side of the anchoring portion 14.

The connection pegs 13b have at least a porous surface suitable for osseointegration externally.

According to an embodiment, illustrated in FIG. 12, the connection pegs 13b can have a porous structure only externally and a compact structure having a reinforcing function internally.

The connection pegs 13b can thus have a mixed structure, porous externally and full internally.

In such a case, the connection pegs 13b have an inner reinforcing part, or core, 13b′, made of compact solid material without a porous structure, which extends, or in other words is part, of the anchoring portion 14, FIG. 12, and an outer porous part 13b″ which extends, or in other words is part, of the interface portion 13.

The inner reinforcing part 13b′ extends only for a part of the length of each connection peg 13b. Therefore, each connection peg 13b′ is completely encased, or surrounded, by the outer porous part 13b″.

The length L of the connection pegs 13b is defined along the insertion axis X between the interface surface 13′ and the tip of the connection pegs 13b. The ratio of the length L1 of the inner reinforcing part 13b′ to the length L of the connection peg 13b can be between about 60% and about 100%.

Furthermore, the ratio of the volume of the inner reinforcing portion 13b′ to the overall volume of the connection peg 13b can be from about 5% to about 50%. The volume of the connection peg 13b is considered from the interface surface 13′ to the tip thereof. The volume of the inner reinforcing part 13b′ is also considered starting from the interface surface 13′.

According to possible embodiments not shown, the inner reinforcing part 13b′ can extend along the entire length of each connection peg 13b.

In another embodiment, the connection pegs 13b can have a completely porous structure. In such a case, the connection pegs 13b are completely part of the interface portion 13.

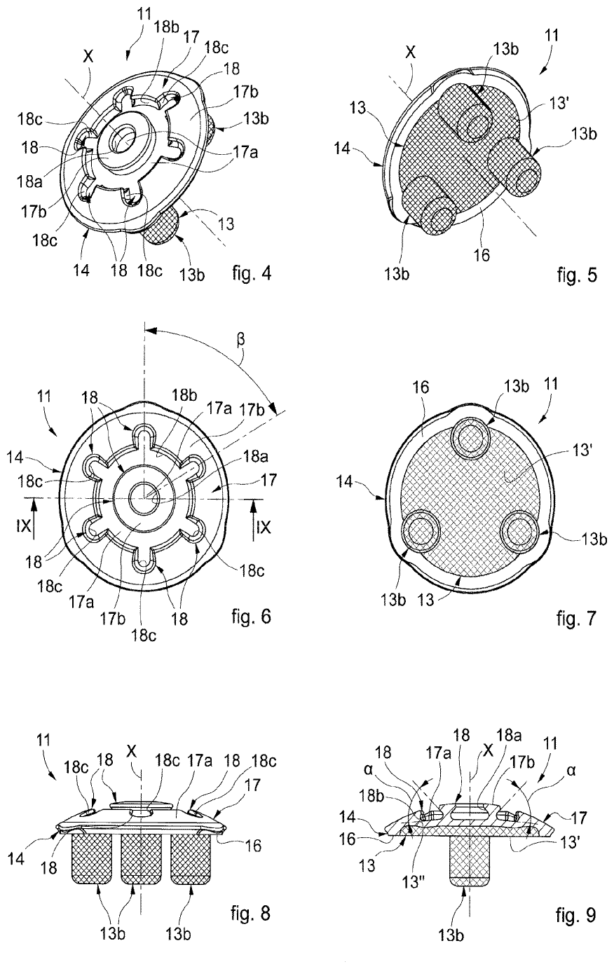

The connection pegs 13b have an exemplary cylindrical shape, see for example FIGS. 5 and 7. Further embodiments are however possible.

The interface portion 13 and the connection pegs 13b are adapted to contact a bone surface 112 of the patella 100 of a patient for osseointegration. In particular, the connection pegs 13b are adapted to engage corresponding holes prepared in the patella 100 parallel to the insertion axis X while the interface surface 13′ rests on the bone surface 112.

According to possible embodiments, the porous structure of the interface portion 13 can have the same or even different features with respect to the porous structure of the connection pegs 13b. One of the distinguishing features can be a porosity gradient, a density value, a permeability value, or other. For example, the porosity of the interface portion 13 and the connection pegs 13b can be homogeneous and constant. Furthermore, the sizes (or nominal diameter) of the pores, or base cells, of the interface portion 13 and the connection pegs 13b can be equal, on average, to about 640 μm.

According to embodiments, the connection pegs 13b can be in partial continuity even with the anchoring portion 14, FIGS. 5 and 7. As can be seen in FIGS. 5 and 7, being located in a peripheral position, the anchoring pegs 13b are also in contact with the anchoring portion 14 in the case of both a mixed, porous and full structure, and in the case of a completely porous structure.

The anchoring portion 14 extends from the interface portion 13, on the opposite side with respect to that from which the connection pegs 13b extend. In particular, the anchoring portion 14 extends from a connection surface 13″ of the interface portion 13 opposite the interface surface 13′, FIGS. 3 and 9.

The anchoring portion 14 has an overall convex shape and wraps the interface portion 13, FIGS. 3, 5 and 9, above and laterally. The anchoring portion 14 has a cap shape.

The anchoring portion 14 has a connection surface 16 substantially conjugated to the shape of the interface portion 13 and opposite the anchoring surface 17 to which the joint cap 12 is anchored, FIG. 3. The anchoring is of macro type. The term “macro anchoring” is intended to mean an anchoring other than a micro fixing, as in the prior art, in which the joint cap 12 is interconnected to the anchoring portion 14 through a porous structure.

The anchoring surface 17 lacks a porous structure and is provided with one or more recesses 18 each having anchoring undercuts to facilitate the irremovable anchoring of the polymeric material of the joint cap 12, FIGS. 3, 4, 6, 8, 10, 11.

As described above, the recesses 18 obtain a macro-anchoring of the joint cap 12 to the support component 11, in particular of the polymeric material to the anchoring portion 14 of the support component 11, unlike the solutions known in the prior art in which a micro fixation is obtained by infusing the polymer into a finely porous structure of the support component, with the disadvantages that this entails, in particular by requiring specific expedients to be taken, for example intermediate and additional compact barrier layers, to block the polymer, preventing it from flowing through the porous structure of the osseointegration interface portion, contacting the bone regrowth therein.

The joint cap 12 is obtained by injection-moulding a polymeric material over the support component 11, in particular on the anchoring portion 14. The polymeric material not yet solidified fills the undercut recesses 18 determining, as a final result, that the joint cap 12 becomes integral with the support component 11.

The anchoring portion 14 is solid/compact in nature, i.e., non-porous, both to provide adequate structural integrity to the patellar prosthesis 10, and to prevent the passage of the polymer through the interface portion 13 and through the connection pegs 13b which have at least a partially reticular structure. I.e., it acts as a barrier to the polymeric material during the moulding step thereof, to prevent the polymeric material from filling the cavities of the reticular structure preventing an effective bone regrowth downstream of the intervention.

The joint cap 12 wraps the anchoring portion 14 above and laterally without contacting the interface portion 13 and the connection pegs 13b, FIG. 3. In other words, the anchoring portion 14 is interposed between the joint cap 12 and the interface portion 13. Therefore, the anchoring of the joint cap 12 is obtained only by the particular shape of the anchoring surface 17.

The recesses 18 extend between a base area 17a of the anchoring surface 17 and a raised area 17b of the anchoring surface 17. FIGS. 3, 4, 6, 10, 11. Therefore, the recesses 18 are open and all communicate directly with the outside.

The walls defining the recesses 18 between the raised area 17b and the base area 17a obtain a acute anchoring angles with the base portion 17a, FIG. 9. Such a configuration of the recesses 18 defines the aforesaid anchoring undercuts.

According to embodiments, the recesses 18 comprise a central recess 18a of circular shape, a peripheral recess 18b of annular shape which extends around the central recess 18a and a plurality of radial recesses 18c which extends radially from the peripheral recess 18b, FIGS. 4, 6 and 8. The presence of the radial recesses 18c allows to constrain the rotation of the joint cap 12 which therefore remains locked in a stable position with respect to the connection cap 14.

According to embodiments, the radial recesses 18c communicate on one side with the peripheral recess 18b and are closed radially on the opposite side, FIGS. 4, 6, and 8. In the example of FIGS. 4 and 6 there are six radial recesses 18c angularly equispaced which substantially define radial inlets.

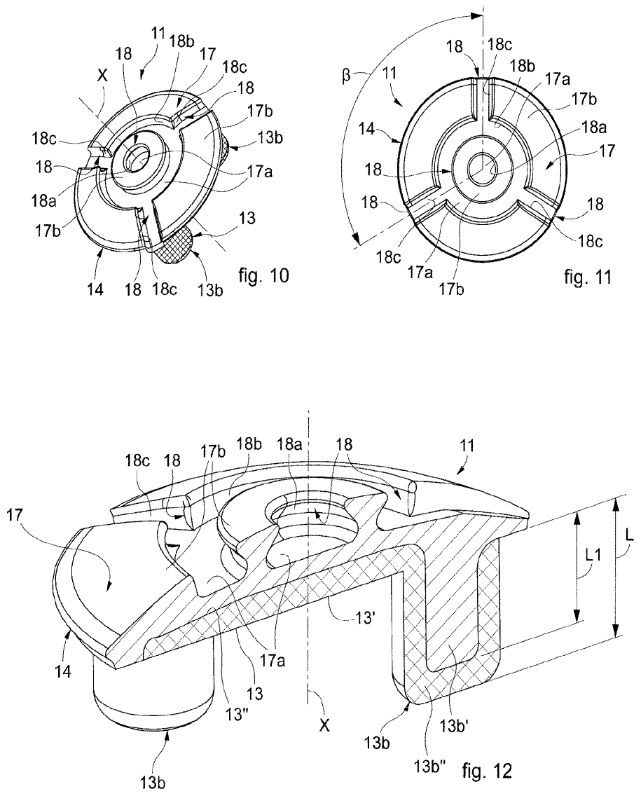

According to further embodiments, the radial recesses 18c communicate on one side with the peripheral recess 18b and are open radially outwards on the opposite side, FIGS. 10-11. In the example of FIGS. 10-11 there are three radial recesses 18c angularly equispaced, substantially defining radial grooves.

The radial recesses 18c are angularly spaced according to a constant pitch angle β. With reference to FIG. 4 the pitch angle β is 60° while in FIG. 11 the pitch angle β is 120°.

According to a variant, the pitch angle β can be variable, i.e., not be equal between one radial recess 18c and the next.

According to embodiments, the volume of the recesses 18 is 2-3 orders of magnitude greater with respect to the volume of the pores, or base cells, of the interface portion 13 and of the connection pegs 13b.

It is clear that modifications and/or additions of parts may be made to the patellar prosthesis 10 described so far, without departing from the scope of the present invention as defined in the claims.

It is also clear that, although the present invention has been described with reference to some specific examples, a person skilled in the art will be able to make many other equivalent forms of patellar prostheses having the features expressed in the claims and therefore all of which falling within the scope of protection defined thereby.

In the following claims, the references in parentheses have the sole purpose of 10 facilitating reading and must not be considered as limiting factors of the scope of protection defined by the claims themselves.

1. Patellar prosthesis (10), comprising:
a support component (11) made of monolithic metallic material and made in a single piece by means of additive manufacturing, provided with an anchoring portion (14) and an interface portion (13) for the osseointegration obtained in direct continuity with said anchoring portion (14), a plurality of connection pegs (13b) extending from said interface portion (13), and
a joint cap (12) in polymeric material, anchored to said anchoring portion (14), wherein said anchoring portion (14) is fully compact and lacks a porous structure and has an anchoring surface (17) provided with one or more recesses (18) each having anchoring undercuts for said polymeric material,
wherein, in addition, said interface portion (13) consists of a completely porous structure suitable for osseointegration.

2. Patellar prosthesis (10) as in claim 1, wherein said anchoring surface (17) is convex and has a base area (17a) and a raised area (17b) between which said recesses (18) extend.

3. Patellar prosthesis (10) as in claim 2, wherein said recesses (18) are delimited by walls defined between said raised area (17b) and said base area (17a) and which create acute anchoring angles (a) with said base portion (17a).

4. Patellar prosthesis (10) as in claim 1, wherein recesses (18) comprise a central recess (18a) of circular shape, a peripheral recess (18b) of annular shape which extends around said central recess (18a) and a plurality of radial recesses (18c) which extend radially from said peripheral recess (18b).

5. Patellar prosthesis (10) as in claim 4, wherein said radial recesses (18c) are angularly spaced according to a constant pitch angle (β).

6. Patellar prosthesis (10) as in claim 4, wherein said radial recesses (18c) communicate on one side with said peripheral recess (18b) and on the opposite side are closed radially.

7. Patellar prosthesis (10) as in claim 4, wherein said radial recesses (18c) communicate on one side with said peripheral recess (18b) and on the other side are open radially outwards.

8. Patellar prosthesis (10) as in claim 1, wherein said connection pegs (13b) have a completely porous structure so that said connection pegs (13b) are completely part of said interface portion (13).

9. Patellar prosthesis (10) as in claim 1, wherein said connection pegs (13b) have an inner reinforcing part, or core, (13b′) in compact solid material lacking a porous structure which is part of said anchoring portion (14) and an outer porous part (13b″) which is part of said interface portion (13).

10. Patellar prosthesis (10) as in claim 9, wherein the ratio between a length (L1) of said inner reinforcing part (13b′) and a length L of said connection peg (13b) is between 60% and 100%.
